(19) United States
(12) Patent Application Publication
(10) Pub. No.: US 2019/0144452 A1
(43) Pub. Date: May 16, 2019

(54) PROCESS FOR PREPARING IDELALISIB

(71) Applicant: Synthon B.V., Nijmegen (NL)

(72) Inventors: Libor VYKLICKY, Blansko (CZ); Zuzana BEDNAROVA, Blansko (CZ); Radomir SKOUMAL, Blansko (CZ)

(21) Appl. No.: 16/188,970

(22) Filed: Nov. 13, 2018

(30) Foreign Application Priority Data
Nov. 10, 2017 (EP) 17201098.5

(51) Int. Cl. C07D 473/34; C07C 271/22

(52) U.S. Cl. CPC C07D 473/34; C07C 271/22

(57) ABSTRACT

The present invention relates to an improved process of preparation of idelalisib using a solid form of intermediate of formula (2);

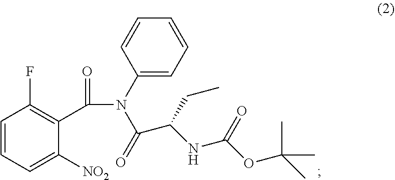 [SMILES: CC[C@H](NC(=O)OC(C)(C)C)C(=O)N(C(=O)c1c(F)cccc1[N+](=O)[O-])c1ccccc1]

characterized by XRPD pattern having 2θ values 5.5°, 10.9°, 14.3°, 16.9° (±0.2) the process comprising steps
a. Reacting of compound of formula (1);

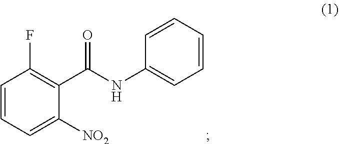 [SMILES: O=C(Nc1ccccc1)c1c(F)cccc1[N+](=O)[O-]]

with a chlorinating agent to obtain a product;
b. Reacting of the product with (S)-2-((tert-butoxycarbonyl)amino)butanoic acid and a base, wherein pH of the reaction mixture is set between 7.5 and 10 to obtain compound of formula (2);
c. Isolating a solid form of compound of formula (2);

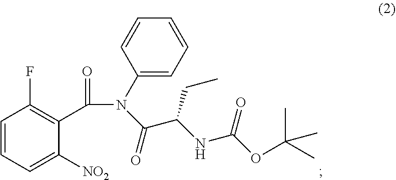 [SMILES: CC[C@H](NC(=O)OC(C)(C)C)C(=O)N(C(=O)c1c(F)cccc1[N+](=O)[O-])c1ccccc1]

d. Transforming the compound of formula (2) into idelalisib.

### BACKGROUND OF THE PRESENT INVENTION

This invention relates to an improved process of preparation of idelalisib using a solid form of intermediate of formula (2);

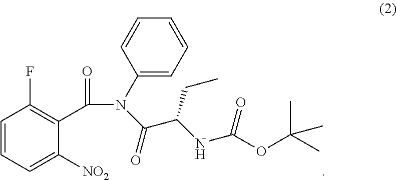 [SMILES: CC[C@H](NC(=O)OC(C)(C)C)C(=O)N(C(=O)c1c(F)cccc1[N+](=O)[O-])c1ccccc1]

### BRIEF DESCRIPTION OF THE PRESENT INVENTION

Idelalisib is a PI3Kδ inhibitor of formula;

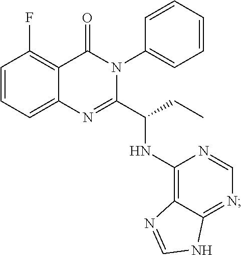 [SMILES: CC[C@H](Nc1ncnc2[nH]cnc12)c1nc2cccc(F)c2c(=O)n1-c1ccccc1]

and is used for the treatment of patients with follicular lymphoma, relapsed small lymphocytic lymphoma and relapsed chronic lymphocytic leukaemia.

Idelalisib was first disclosed in WO2005113556 application.

WO2005113556 describes the preparation of idelalisib as depicted below:

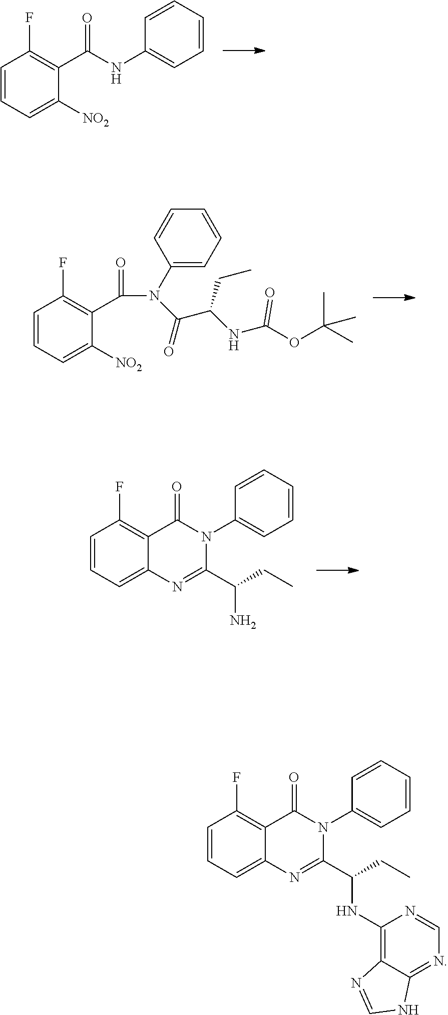 [SMILES: O=C(Nc1ccccc1)c1c(F)cccc1[N+](=O)[O-] ; CC[C@H](NC(=O)OC(C)(C)C)C(=O)N(C(=O)c1c(F)cccc1[N+](=O)[O-])c1ccccc1 ; CC[C@H](N)c1nc2cccc(F)c2c(=O)n1-c1ccccc1 ; CC[C@H](Nc1ncnc2[nH]cnc12)c1nc2cccc(F)c2c(=O)n1-c1ccccc1]

The process for preparing idelalisib as described in WO2005113556 requires chromatographic purification steps for several intermediates and for idelalisib. The overall yield of the depicted process is 20%.

A modification of the WO2005113556 process is described in WO2016108206. The overall yield of the process described in WO2016108206 (based on 2-fluoro-6-nitro-N-phenylbenzamide) is 19% of the theoretical yield and the process also comprises chromatographic purification of an intermediate.

Chromatographic purification steps are tedious and expensive process steps on an industrial scale. Therefore, there is a need for alternative processes that do not comprise chromatographic purification and with improved yield of idelalisib.

### SUMMARY OF THE INVENTION

The presented invention relates to a process for preparing idelalisib of formula:

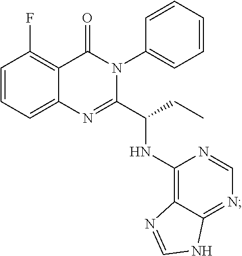 [SMILES: CC[C@H](Nc1ncnc2[nH]cnc12)c1nc2cccc(F)c2c(=O)n1-c1ccccc1]

the process comprising:
a. Reacting of compound of formula (1);

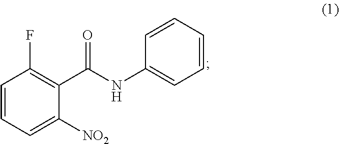 [SMILES: O=C(Nc1ccccc1)c1c(F)cccc1[N+](=O)[O-]]

with a chlorinating agent to obtain a product;
b. Reacting of the product with (S)-2-((tert-butoxycarbonyl)amino)butanoic acid and a base, wherein pH of the reaction mixture is set between 7.5 and 10 to obtain compound of formula (2);
c. Isolating a solid form of compound of formula (2);

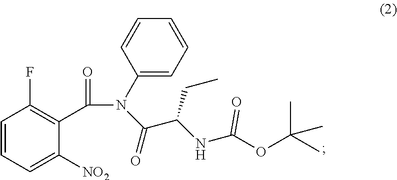 [SMILES: CC[C@H](NC(=O)OC(C)(C)C)C(=O)N(C(=O)c1c(F)cccc1[N+](=O)[O-])c1ccccc1]

wherein the solid compound of formula (2) is characterized by XRPD pattern having 2θ values 5.5°, 10.9°, 14.3°, 16.9° (±0.2);
d. Transforming the compound of formula (2) into idelalisib.

### BRIEF DESCRIPTION OF THE DRAWING

The sole FIGURE depicts the XRPD pattern of a solid form of the compound of formula (2).

### DETAILED DESCRIPTION OF THE INVENTION

The presented invention relates to a process for preparing idelalisib of formula:

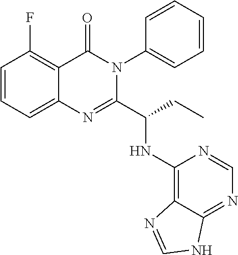 [SMILES: CC[C@H](Nc1ncnc2[nH]cnc12)c1nc2cccc(F)c2c(=O)n1-c1ccccc1]

the process comprising:
a. Reacting of compound of formula (1);

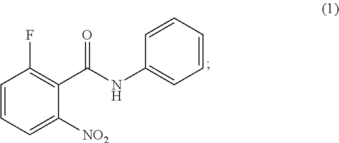 [SMILES: O=C(Nc1ccccc1)c1c(F)cccc1[N+](=O)[O-]]

with a chlorinating agent to obtain a product
b. Reacting of the product with (S)-2-((tert-butoxycarbonyl)amino)butanoic acid and a base, wherein pH of the reaction mixture is set between 7.5 and 10 to obtain compound of formula (2);
c. Isolating a solid form of compound of formula (2):

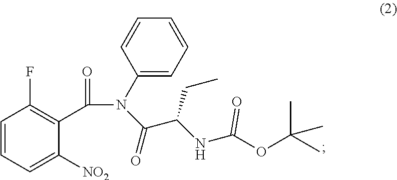 [SMILES: CC[C@H](NC(=O)OC(C)(C)C)C(=O)N(C(=O)c1c(F)cccc1[N+](=O)[O-])c1ccccc1]

wherein the solid compound of formula (2) is characterized by XRPD pattern having 2θ values 5.5°, 10.9°, 14.3°, 16.9° (±0.2);
d. Transforming the compound of formula (2) into idelalisib.

The isolating step c. optionally comprises following steps
1. Concentrating of the reaction mixture;
2. Adding to the rest a solvent selected from C1-C8 alcohols or a mixture thereof;
3. Heating the mixture to a temperature between 35° C. and reflux temperature of the solvent to dissolve the rest;
4. Cooling the mixture to a temperature between −20° C. and the 35° C.;
5. Isolating the solid form of compound of formula (2).

The chlorinating agent in the reaction step a. can be for example thionyl chloride. The chlorination reaction is done in the presence of N,N-dimethylformamide (DMF). The molar ratio between compound (1) and the chlorinating agent can be between 1:2 and 1:10, preferably between 1:2 and 1:5, more preferred 1:3.5. The reaction step a. can optionally be done in a suitable solvent. The reaction temperature can be between 60° C. and the reflux temperature of used solvents, preferably it is between 70° C. and 85° C. The reaction time can be between 1 and 10 hours, preferably between 1 and 5 hours. During the reaction the reaction mixture can be protected from the humidity, for example by using nitrogen atmosphere. The reaction progress can be monitored by a suitable analytical technique, e.g. by HPLC or GC. When the reaction is finished the chlorinating agent and used solvents are distilled off to obtain the chlorinated product.

A first solution is prepared by dissolving the chlorinated product and a base in a suitable solvent, for example dichloromethane or chloroform or tetrahydrofurane (THF) or an ether (for example tert-butyl ether, diethyl ether) or toluene or a mixture thereof. Dichloromethane is preferably used. The concentration of the chlorinated product in the solvent can be between 0.3 g/ml and 3 g/ml, more preferably between 0.5 and 1 g/ml. The molar ratio between the chlorinated product and the base can be between 1:1 and 1:10, preferably 1:2. pH of the first solution is set between 7.5 and 10, more preferably between 8 and 9 using a base. The base can be an organic base (for example amines like triethylamine, diethylamine, pyridine, piperidine, diethylpropylamine) or an inorganic base (for example alkaline metal carbonates or alkaline metal hydrocarbonates or alkali metal or alkaline metal hydroxides) can be used. Preferably an amine, more preferably triethylamine is used.

A second solution is prepared by dissolving (S)-2-((tert-butoxycarbonyl)amino)butanoic acid and a base in a suitable solvent, for example dichloromethane or chloroform or tetrahydrofurane (THF) or an ether (for example tert-butyl ether, diethyl ether) or toluene or a mixture thereof is prepared. Dichloromethane is preferably used. The concentration of (S)-2-((tert-butoxycarbonyl)amino)butanoic acid in the solvent can be between 0.2 g/ml and 1 g/ml, more preferably between 0.3 and 0.6 g/ml. The molar ratio between (S)-2-((tert-butoxycarbonyl)amino)butanoic acid and the base can be between 1:1 and 1:2, preferably 1:1.1. pH of the second solution is set between 7.5 and 10, more preferably between 8 and 9 using a base. The base can be an organic base (for example amines like triethylamine, diethylamine, pyridine, piperidine, diethylpropylamine) or an inorganic base (for example alkaline metal carbonates or alkaline metal hydrocarbonates or alkali metal or alkaline metal hydroxides) can be used. Preferably an amine, more preferably triethylamine is used.

Both solutions are mixed together. The molar ratio between (S)-2-((tert-butoxycarbonyl)amino)butanoic acid and the chlorinated product can be between 1:1 and 1:5, more preferably between 1:1 and 1:1.5. The reaction is done at a temperature between 15° C. and reflux temperature of the solvent, preferably at 25° C. The reaction time is between 1 and 7 hours, preferably it is between 1 and 2 hours. The reaction progress might be monitored by a suitable analytical technique, e.g. by HPLC or GC.

We have surprisingly found that when pH of the reaction mixture in the reaction step b. is set between 7.5 and 10, preferably between 8 and 9, the purity of obtained compound of formula (2) is higher comparing to the purity of compound of formula (2) prepared by a prior art process.

The base to adjust pH can be added in the solutions before mixing them together, as described above, it can be also added into the reaction mixture in the beginning of the reaction after the first and the second solutions are mixed or in the course of the reaction to maintain the pH of the mixture between 7.5 and 10.

The pH is set up by any suitable base. The base can be an organic base (for example amines like triethylamine, diethylamine, pyridine, piperidine, diethylpropylamine) or an inorganic base (for example alkaline metal carbonates or alkaline metal hydrocarbonates or alkali metal or alkaline metal hydroxides) can be used. Preferably an amine, more preferably triethylamine is used.

After the reaction is finished the mixture can be cooled for example to the room temperature and can be extracted by for example water. The reaction mixture can be treated with activated carbon or cellite and filtrated. The filtrated mixture can be concentrated.

We have also surprisingly found that the compound of formula (2) can be isolated in a solid form characterized by XRPD pattern having 2θ values 5.5°, 10.9°, 14.3°, 16.9° (±0.2). The isolation of the solid form of compound of formula (2) together with adjustment of pH of the reaction mixture described above results in a process for idelalisib preparation where no chromatographic purification is needed and improves the overall yield of the whole process.

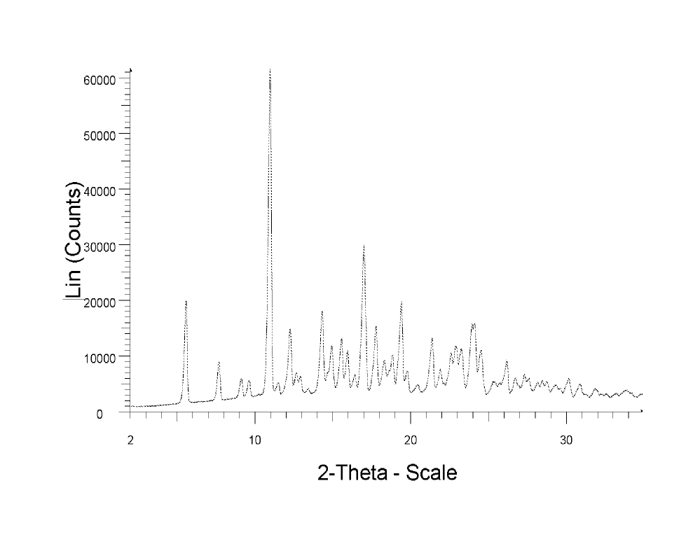

The invention therefore also relates to a solid form of compound of formula (2);

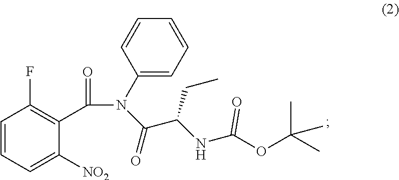 [SMILES: CC[C@H](NC(=O)OC(C)(C)C)C(=O)N(C(=O)c1c(F)cccc1[N+](=O)[O-])c1ccccc1]

wherein the solid form is characterized by XRPD pattern having 2θ values 5.5°, 10.9°, 14.3°, 16.9° (±0.2). The solid form of compound of formula (2) is further characterized by XRPD spectrum depicted in FIG. 1. The solid form of compound of formula (2) is also characterized by XRPD pattern having 20 values:

| Angle 2-Theta° | Intensity % |
|---|---|
| 5.5 | 32.2 |
| 7.6 | 14.3 |
| 9.1 | 9.4 |
| 9.6 | 8.7 |
| 10.9 | 100.0 |
| 11.4 | 8.2 |
| 12.2 | 24.0 |
| 12.6 | 11.0 |
| 12.9 | 10.1 |
| 14.3 | 29.1 |
| 14.9 | 19.0 |
| 15.5 | 21.3 |
| 15.9 | 17.5 |
| 16.4 | 10.5 |
| 16.9 | 48.4 |
| 17.7 | 24.6 |
| 18.3 | 14.7 |
| 18.8 | 16.3 |
| 19.4 | 31.7 |
| 19.7 | 11.8 |
| 20.4 | 7.5 |
| 21.3 | 21.2 |
| 21.9 | 12.2 |
| 22.6 | 16.9 |
| 22.9 | 18.9 |
| 23.2 | 18.2 |
| 24.1 | 25.3 |
| 24.5 | 17.4 |
| 25.3 | 8.6 |
| 25.7 | 8.2 |
| 26.1 | 14.9 |
| 26.7 | 9.8 |
| 27.3 | 10.7 |
| 27.5 | 9.3 |
| 28.1 | 8.4 |
| 28.4 | 8.8 |
| 28.7 | 8.5 |
| 29.3 | 7.6 |
| 29.6 | 6.7 |
| 30.1 | 9.5 |
| 30.9 | 7.8 |

The solid form of compound of formula 2 can be obtained by dissolving compound of formula (2) in a solvent selected from C₁-C₈ alcohol or a mixture thereof. Advantageously water can be added into the mixture. The alcohol is preferably selected from ethanol or 2-propanol or 1-butanol or 2-butanol or tert-butanol or a mixture thereof. Preferably a mixture of ethanol and 2-propanol is used and the ratio between ethanol:2-propanol is between 1:3.5 to 1:6.5 (vol:vol), more preferably the ratio is 1:5 (vol:vol). Advantageously water can be added into the mixture. The ration between the alcohol or a their mixture and water can be between 2.5:2 and 1:1 (alcohol:water, vol:vol:vol), preferably it is 1:1 (vol:vol:vol). In a preferred embodiment a mixture of ethanol, 2-propanol and water is used and the ratio between ethanol:2-propanol:water (vol:vol:vol) is between 1:1.5:2 and 1:3:4, more preferably it is 1:2:3. The concentration of compound of formula (2) in an alcohol or a mixture thereof is between 0.05 g/ml and 0.5 g/ml, preferably between 0.1 g/ml and 0.4 g/ml, most preferably 0.2 g/ml. The mixture of compound of formula (2) in the alcohol solvent or a mixture of alcohol solvents can be heated before compound of formula (2) is crystallized. The mixture is heated to a temperature between 35° C. and reflux temperature of the solvent. The mixture is preferably heated to 45-70° C. In the case that water is optionally added to the mixture, it is added slowly, for example during 20, 30, 40, 50 or 60 minutes. Preferably, water is added to the mixture after heating of the mixture. The mixture is then cooled to a temperature between −20° C. and the room temperature to crystallize compound of formula (2) from the mixture. The temperature is preferably between −10° C. and the room temperature (20-25° C.). Solid compound of formula (2) can be isolated from the mixture using any isolation techniques, for example by filtration. The isolated solid mass can be optionally washed and dried.

The obtained solid compound of formula (2) show a good purity, crystallinity and stability and can be used for purification of compound of formula 2 or in the process for preparation of idelalisib of formula (1) or structurally related compounds.

Starting 2-fluoro-6-nitro-N-phenylbenzamide can be prepared by a process described in WO2005113556 application.

XRPD spectra were obtained using the following measurement conditions: Bruker-AXS D8 Vario diffractometer with Θ/2Θ geometry (reflection mode), equipped with a Vantec PSD detector.

| | |
|---|---|
| Start angle (2θ): | 2.0° |
| End angle (2θ): | 35.0° |
| Scan step width: | 0.02° |
| Scan step time: | between 0.2-2.0 seconds |
| Radiation type: | Cu |
| Radiation wavelengths: | 1.5406 Å (Kα1), primary monochromator used |
| Exit slit: | 6.0 mm |
| Focus slit: | 0.2 mm |
| Divergence slit: | Variable (V20) |
| Antiscatter slit: | 11.8 mm |
| Receiving slit: | 20.7 mm |

The invention will be further illustrated by the following, non-limiting, examples.

### EXAMPLES

### Example 1

Preparation of Idelalisib:

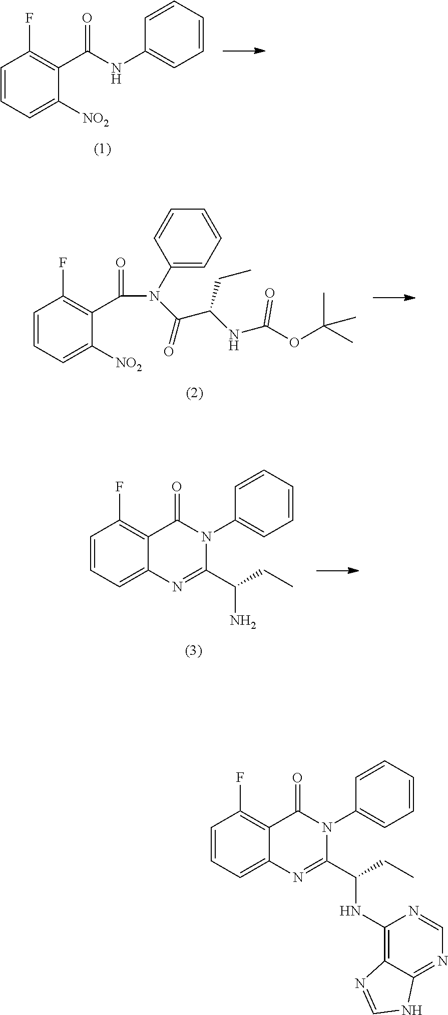 [SMILES: O=C(Nc1ccccc1)c1c(F)cccc1[N+](=O)[O-] ; CC[C@H](NC(=O)OC(C)(C)C)C(=O)N(C(=O)c1c(F)cccc1[N+](=O)[O-])c1ccccc1 ; CC[C@H](N)c1nc2cccc(F)c2c(=O)n1-c1ccccc1 ; CC[C@H](Nc1ncnc2[nH]cnc12)c1nc2cccc(F)c2c(=O)n1-c1ccccc1]

### Example 1.1: Preparation of Compound of Formula (2)

50 g of 2-fluoro-6-nitro-N-phenylbenzamide (1) was suspended in 80 g of SOCl₂ and 1.5 g of dimethylformamide. The mixture was refluxed at 70° C. for 1 hour under nitrogen atmosphere. The reaction mixture was evaporated at 85° C. under reduced pressure. The rest was dissolved in 80 g of dichloromethane and the mixture was concentrated.

The rest was dissolved in 120 g of dichloromethane and 7.3 g of dry triethylamine were added. pH of the solution was set up at 8 (at 20-25° C.) using triethylamine. pH was measured using water wetted pH indicator strips.

43 g of (S)-2-((tert-butoxycarbonyl)amino)butanoic acid was mixed with 150 g of dichloromethane and 21.5 g of dry triethylamine to obtain a second solution. pH of the second solution was set up at 8 (at 20-25° C.) using triethylamine. pH was measured using water wetted pH indicator strips.

Both solutions were mixed together. The mixture was stirred at 25° C. for 1.5 hour. The mixture was concentrated. To the rest 150 g of ethylacetate and 240 g of water were added. The mixture was stirred at 65° C. for 10 minutes, the mixture was cooled at 20° C. and the phases were separated. The water phase was extracted with 30 g of ethylacetate. Phases were separated, the organic phases were collected and concentrated.

### Example 1.2: Crystallization of Compound of Formula (2) from Mixture 2-Propanol/Ethanol/Water

To the rest 180 g of 2-propanol and 95 g of ethanol were added. The mixture was stirred at 45-50° C. for 10 minutes. To the mixture 270 g of water was added in the course of 30 minutes. To the mixture 0.3 g of compound (2) seed prepared according to example 4 was added. The mixture was stirred at 45-50° C. for 30 min and it was cooled to 0° C. It was stirred at this temperature for 60 minutes and filtered off. Filtrated mass was dried. 57.1 g of Compound (2) was obtained (corresponds to 67% of theoretical yield based on amount of starting 2-fluoro-6-nitro-N-phenylbenzamide) having HPLC purity 94% (HPLC IN). XRPD of the obtained solid corresponds to XRPD depicted in FIG. 1.

### Example 1.3: Preparation of Idelalisib

50 g of compound of formula (2) was mixed with 300 g of acetic acid. The mixture was cooled at 15° C. and 42 g of zinc was slowly added into the mixture in the course of 120 minutes. During the zinc addition the temperature of the reaction mixture is maintained between 10-20° C. After addition of the zinc, the mixture is stirred for additional 2 hours at 20° C. Mixture was filtered off. To the filtrate 65 g of trifluoroacetic acid are added. The mixture is heated at 75° C. and stirred for 1.5 hour. The mixture was distilled off at 100° C.

To the rest 300 g of ethylacetate are added. pH of the mixture was set at 9 (measured by pH meter) using a solution containing 50 g of ammonium hydroxide in 100 g of water.

The phases are separated and the ethylacetate solution was extracted with 150 g of water and the layers were separated. The extraction of the ethylacetate solution was repeated two more times. To the ethylacetate solution 80 g of toluene was added. From the mixture 320 g of ethylacetate-toluene mixture was distilled off. The mixture was cooled to −10° C. and stirred at this temperature for 60 minutes. Compound of formula (3) was filtered off from the mixture to provide 21.7 g of compound of formula (3) (65% of the theoretical yield, based on amount of compound (2)).

15 g of compound (3) was mixed with 14.1 g of 6-bromopurine, 5.4 g of sodium acetate, 3.0 g of acetic acid and 47 g of 2-propanol. The mixture was stirred at 80° C. for 18 hour. Reaction mixture was cooled down, diluted with 150 g of dichloromethane. To the mixture 2.5 g of kieselguhr was added. The mixture was filtered off, the filtrated mass was washed with 75 g of dichloromethane. The filtrate was subsequently washed with 75 g of 10% water solution of acetic acid, 75 g of water, 75 g of 10% water solution of Na₂CO₃ and 75 g of water. The organic layer was dried over MgSO₄, filtered off and concentrated to provide 16.4 g of idelalisib (yield 78% based on compound (3)).

The overall yield based on starting 2-fluoro-6-nitro-N-phenylbenzamide is 34% of the theoretical yield. The purity of isolated solid was 92% (HPLC IN).

### Example 2: Preparation of Idelalisib—Comparative Example (Examples 24-28, WO2016108206)

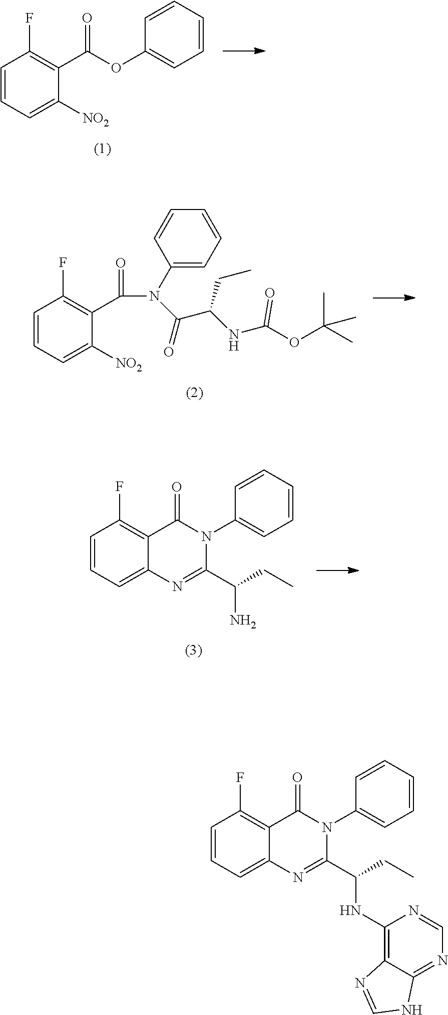 [SMILES: O=C(Oc1ccccc1)c1c(F)cccc1[N+](=O)[O-] ; CC[C@H](NC(=O)OC(C)(C)C)C(=O)N(C(=O)c1c(F)cccc1[N+](=O)[O-])c1ccccc1 ; CC[C@H](N)c1nc2cccc(F)c2c(=O)n1-c1ccccc1 ; CC[C@H](Nc1ncnc2[nH]cnc12)c1nc2cccc(F)c2c(=O)n1-c1ccccc1]

The mixture of 23 g of compound of formula (1), 45.15 ml of thionyl chloride and 0.3 ml of N,N-dimethylformamide was heated to 85° C. and maintained for 3 hours at 85° C. The reaction mixture was concentrated completely under vacuum and the residue was dissolved in 60 ml of dichloromethane. 25.14 g of N-Boc-L-2-aminobutyric acid, 100 ml of dichloromethane and 18.48 ml of triethylamine were charged and the mixture was cooled to 5° C. The imidoyl chloride solution was added drop-wise into the N-Boc-L-2-aminobutyric acid solution over a period of 20 minutes and the mixture was stirred for 20 hours at 30° C. 300 ml of dichloromethane and 200 ml of water were added to the reaction mixture and stirred for 10 minutes. Organic layer was separated and was washed with 120 ml of saturated NaHCO₃ solution, 100 ml of 10% citric acid solution, 150 ml of water and 100 ml of brine. The organic layer dried over 20 g of anhydrous sodium sulphate and concentrated completely under reduced pressure to yield 40 g of the crude product of formula (2).

A mixture of 40 g of compound of formula (2) and 400 ml of acetic acid was cooled to 15° C. 35.22 g of activated Zinc powder was added portion wise over a period of 30 minutes and the mass was stirred for 20 hours at 20° C. The reaction mass was filtered through a celite bed and the bed was washed with 200 ml of acetic acid. The mass was concentrated completely under reduced pressure and the residue was dissolved in 300 ml of water. The solution was basified with 70 g of solid NaHCO₃ and extracted with 2×400 ml of ethylacetate. The organic layer was extracted with 2×300 ml of water and 200 ml of brine and dried over anhydrous sodium sulphate and concentrated completely under reduced pressure to yield 31 g of crude product.

31 g of the crude product and 31 ml THF were charged into a 1 L round bottom flask and the mixture was cooled to 10° C. 62 ml of concentrated HCl was added drop-wise over a period of 15 minutes and the reaction mass was stirred for 2 hours at 30° C. 100 ml of water was added and the resulted solution was washed with 200 mL of ethylacetate and n-hexane mixture (1:1). The aqueous layer was basified with 70 g of solid K₂CO₃ and extracted with 2×300 ml of ethylacetate. The organic layer was washed with 2×200 ml of water and 100 ml of brine and dried over anhydrous sodium sulphate and concentrated completely under reduced pressure. The crude product was purified by column chromatography using silica gel (100-200 mesh) (MeOH, DCM, TFA mobile phase). Eluted pure fractions were evaporated and dissolved in 150 ml water and basified with 15 g of solid K₂CO₃ and extracted with 2×200 ml ethylacetate. The organic layer was washed with 150 ml water and 100 ml brine solution, dried over anhydrous sodium sulphate and concentrated completely under reduced pressure to yield 9.5 g of the product.

To 9.5 g of the product in 31 ml THF 11.46 g dibenzoyl-L-tartaric acid was added and the mixture was heated to 55° C. and stirred for 30 minutes. The reaction mass was diluted with 200 ml MTBE and stirred for 10 hours at 30° C. The reaction mass was filtered and washed with 100 ml MTBE and concentrated completely under vacuum. The resulted crude was dissolved in 200 ml water and basified with 10 g of solid K₂CO₃. The mixture extracted with 3×200 ml of ethylacetate. The organic layer was washed with 100 ml water and 100 ml brine, dried over anhydrous sodium sulphate and concentrated completely under reduced pressure to yield 8.5 g of off-white solid (compound (3)).

To the mixture of 8.5 g of compound (3) and 42 ml t-butanol 7.8 ml of triethylamine and 5 g of 6-chloropurine were added at 30° C. The resultant reaction mixture was heated to 85° C. and stirred for 24 hours. The reaction mixture was evaporated completely under reduced pressure at 40° C. The resultant residue was diluted with 200 ml water and stirred for 30 minutes. The precipitate was filtered and the solid was washed with 60 ml water and 100 ml n-hexane and dried for 1 hour under vacuum to obtain 7 g of idelalisib. The overall yield based on starting 2-fluoro-6-nitro-N-phenylbenzamide is 19% of the theoretical yield. The purity of isolated solid was 61% (HPLC IN).

### Example 3: Crystallization of Compound of Formula (2) from a Mixture Ethanol/2-Propanol

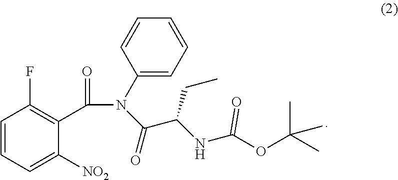 [SMILES: CC[C@H](NC(=O)OC(C)(C)C)C(=O)N(C(=O)c1c(F)cccc1[N+](=O)[O-])c1ccccc1]

5 g of compound of formula (2) prepared according to the procedure described in Example 1.1 was dissolved in 10.6 ml of 2-propanol and 2 ml of Ethanol at 65° C. The mixture was stirred for 15 minutes at 65° C. To the mixture 0.3 g of compound (2) seed prepared according to example 4 was added. The solution was cooled at −10° C. and stirred at this temperature for 3 hours. Obtained suspension was filtered off, the filtrated mass was washed with 3 ml of 2-propanol. Compound of formula (2) was obtained in yield 4.4 g (88% of theoretical yield) and purity 95% (HPLC IN). XRPD of the obtained solid corresponds to XRPD depicted in FIG. 1.

### Example 4: Preparation of Compound (2) Seed

5 g of compound of formula (2) prepared according to the procedure described in Example 1.1 was dissolved in 10.6 ml of 2-propanol and 2 ml of Ethanol at 65° C. The mixture was stirred for 15 minutes at 65° C. The solution was cooled at −10° C. and stirred at this temperature for 3 hours. Obtained suspension was filtered off, the filtrated mass was washed with 3 ml of 2-propanol. Compound of formula (2) was obtained yield 4.7 g (94% of theoretical yield) and purity 98% (HPLC IN). XRPD of the obtained solid corresponds to XRPD depicted in FIG. 1.

1. A process for preparing idelalisib of formula: 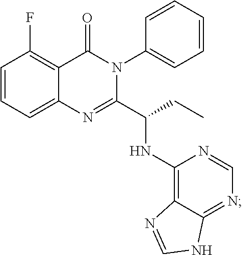 [SMILES: CC[C@H](Nc1ncnc2[nH]cnc12)c1nc2cccc(F)c2c(=O)n1-c1ccccc1]
the process comprising:
a. Reacting of compound of formula (1): 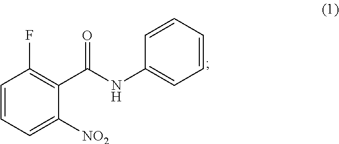 [SMILES: O=C(Nc1ccccc1)c1c(F)cccc1[N+](=O)[O-]]
with a chlorinating agent to obtain a product;
b. Reacting of the product with (S)-2-((tert-butoxycarbonyl)amino)butanoic acid and a base, wherein pH of the reaction mixture is set between 7.5 and 10 to obtain compound of formula (2);
c. isolating a solid form of compound of formula (2); 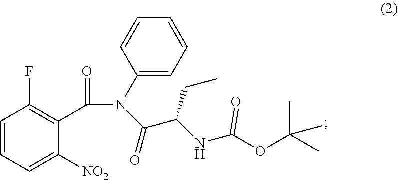 [SMILES: CC[C@H](NC(=O)OC(C)(C)C)C(=O)N(C(=O)c1c(F)cccc1[N+](=O)[O-])c1ccccc1]
wherein the solid compound of formula (2) is characterized by XRPD pattern having 2θ values 5.5°, 10.9°, 14.3°, 16.9° (±0.2); and
d. Transforming the compound of formula (2) into idelalisib.

2. The process according to claim 1, wherein pH of the reaction mixture in step b. is set between 8 and 9.

3. The process according to claim 1, wherein the base in reaction step b. is selected from triethylamine or diethylamine or pyridine or piperidine or diethypropylamine or alkaline metal carbonate or alkali metal hydrogencarbonate.

4. The process according to claim 2, wherein the pH is set by a base selected from triethylamine or diethylamine or pyridine or piperidine or diethypropylamine or alkaline metal carbonate or alkali metal hydrogencarbonate.

5. The process according to claim 1, wherein the solvent in step b. is selected from dichloromethane or chloroform or THF or ethers or toluene or a mixture thereof.

6. The process according to claim 1, wherein a temperature of the reaction mixture in step b. is between 0° C. and 50° C.

7. The process according to claim 1, wherein the step c. comprises:
1. Concentrating of the reaction mixture;
2. Adding to the rest a solvent selected from C1-C8 alcohols or a mixture thereof;
3. Heating the mixture to a temperature between 35° C. and reflux temperature of the solvent to dissolve the rest;
4. Cooling the mixture to a temperature between −20° C. and the 35° C.; and
5. Isolating the solid form of compound of formula (2).

8. The process according to claim 7, wherein the solvent in step II is a mixture of ethanol and 2-propanol and the ratio ethanol:2-propanol is between 1:3.5 and 1:6.5 (vol:vol).

9. The process according to claim 8, wherein the ratio ethanol:2-propanol is 1:5 (vol:vol)

10. The process according to claim 9, wherein the step III further comprises adding water into the mixture.

11. The process according to claim 10, wherein the ratio ethanol:2-propanol:water is between 1:1.5:2 and 1:3:4 (vol:vol:vol).

12. The process according to claim 11, wherein the ratio ethanol:2-propanol:water is 1:2:3 (vol:vol:vol).

13. A solid form of compound of formula (2); 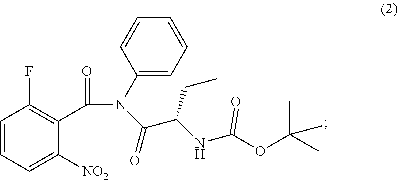 [SMILES: CC[C@H](NC(=O)OC(C)(C)C)C(=O)N(C(=O)c1c(F)cccc1[N+](=O)[O-])c1ccccc1]
wherein the solid form is characterized by XRPD pattern having 2θ values 5.5°, 10.9°, 14.3°, 16.9° (±0.2).

14. The solid form according to claim 13, wherein the solid form is characterized by XRPD pattern having 2θ values 5.5°, 10.9°, 12.2°, 14.3°, 16.9°, 17.7° and 19.4° (±0.2).
